(19) United States
(12) Patent Application Publication
(10) Pub. No.: US 2025/0262348 A1
(43) Pub. Date: Aug. 21, 2025

(54) HIERARCHICALLY POROUS SLIT3-RELEASING PLGA/HYDROXYAPATITE NANONANOCOMPOSITE SCAFFOLD VIA INDIRECT 3D PRINTING FOR BONE TISSUE ENGINEERING

(71) Applicant: University of North Dakota, Grand Forks, ND (US)

(72) Inventor: Ali S. Alshami, Grand Forks, ND (US)

(21) Appl. No.: 19/056,235

(22) Filed: Feb. 18, 2025

Related U.S. Application Data
(60) Provisional application No. 63/553,889, filed on Feb. 15, 2024.

(51) Int. Cl. A61L 27/18; A61L 27/12; A61L 27/34; A61L 27/56; C08L 67/04

(52) U.S. Cl. CPC A61L 27/18; A61L 27/12; A61L 27/34; A61L 27/56; C08L 67/04; A61L 2300/112; A61L 2400/08; A61L 2400/12; A61L 2420/02; A61L 2430/02

(57) ABSTRACT

A polymeric scaffold for bone repair and regeneration includes a body comprising a biodegradable polymer matrix and nanoparticles dispersed in the biodegradable polymer matrix, and a polydopamine surface coating on the body. A method of forming a composite scaffold for bone tissue engineering includes preparing a solution comprising a biodegradable polymer and a plurality of nanoparticles in a solvent, applying the solution to a surface of a mold and drying the solution to form a polymer matrix having nanoparticles dispersed therein, removing the mold from the polymer matrix to form an intermediate scaffold, and applying a polydopamine coating to the intermediate scaffold to form the composite scaffold.

### CROSS-REFERENCE TO RELATED APPLICATION(S)

This application claims the benefit of U.S. Provisional Application No. 63/553,889 filed Feb. 15, 2024 for “HIERARCHICALLY POROUS SLIT3-RELEASING PLGA/HYDROXYAPATITE NANONANOCOMPOSITE SCAFFOLD VIA INDIRECT 3D PRINTING FOR BONE TISSUE ENGINEERING” by A. Alshami.

### BACKGROUND

Recent advances in bone tissue engineering (BTE) have motivated researchers to develop artificial scaffolds that mimic the bone's natural microenvironment, bone extracellular matrix (ECM), and bone-healing mechanisms. These scaffolds need to be able to promote osteogenesis and angiogenesis activities at the defective bone by facilitating cell adhesion, proliferation, migration, communication, and differentiation, and must be biocompatible, bioresorbable, and biodegradable. The current challenges of replicating the extracellular matrix for bone regeneration applications include limited biomaterial options, restricted synthesis approaches, and unreliable behavior of biological stimulators in the scaffold matrix. Further, pore size, porosity ratio, interconnectivity, and mechanical stiffness must be comparable to natural tissue. Achieving these specifications is challenging due to the biological and structural complexity of bone tissue and the limitations of the currently available biomaterials and manufacturing techniques. A range of biomaterials have been investigated for use as potential scaffolding matrices. Despite many of these biomaterials demonstrating highly desirable properties, they have also had significant drawbacks limiting their expanded use for BTE applications.

### SUMMARY

In one aspect, a polymeric scaffold for bone repair and regeneration includes a body comprising a biodegradable polymer matrix and nanoparticles dispersed in the biodegradable polymer matrix, and a polydopamine surface coating on the body.

In another aspect, a method of forming a composite scaffold for bone tissue engineering includes preparing a solution comprising a biodegradable polymer and a plurality of nanoparticles in a solvent, applying the solution to a surface of a mold and drying the solution to form a polymer matrix having nanoparticles dispersed therein, removing the mold from the polymer matrix to form an intermediate scaffold, and applying a polydopamine coating to the intermediate scaffold to form the composite scaffold.

The present summary is provided only by way of example, and not limitation. Other aspects of the present disclosure will be appreciated in view of the entirety of the present disclosure, including the entire text, claims and accompanying figures.

### BRIEF DESCRIPTION OF THE DRAWINGS

FIG. 1 is a is a flow chart of a method for forming SLIT3 protein-releasing composite scaffolds with hierarchically porous structures.

FIG. 2A is a top view of a mold used in the fabrication of the composite scaffold of FIG. 1.

FIG. 2B is a side view of the mold of FIG. 2A.

FIGS. 3A-3D are SEM micrographs of a surface and cross-sectional microstructure of the composite scaffold at different magnifications.

While the above-identified figures set forth embodiments of the present invention, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of the principles of the invention. The figures may not be drawn to scale, and applications and embodiments of the present invention may include features, steps and/or components not specifically shown in the drawings.

### DETAILED DESCRIPTION

An indirect three-dimensional (3D) printing technique is used to produce hierarchically porous Slit Guidance Ligand 3 (SLIT3)-releasing polymeric bone scaffolds (referred to hereinafter as “composite scaffold”) that significantly promote cell adhesion, proliferation, and differentiation. The disclosed composite scaffold exhibits remarkable bone biomimetic properties due to its tailored composition and intricate structure. The present disclosure provides a reliable, controllable, reproducible, and cost-effective fabrication method for developing 3D and hierarchical nanocomposite bone scaffolds. Use of SLIT3 protein as a biological modifier and activator of stem cell proliferation and differentiation instead of cytokines or other traditional proteins within the composite scaffold has proved superior to other synthetic scaffolds. Additionally, the biocompatibility of the SLIT3 protein-releasing composite scaffold provides an attractive platform for future in-vitro and in-vivo biological assessment for bone regeneration applications.

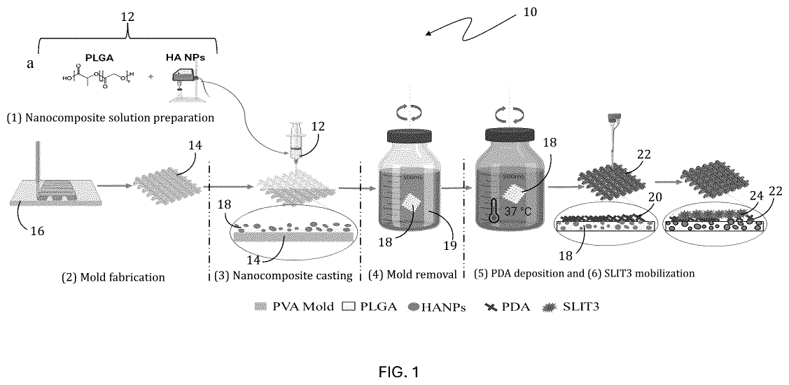

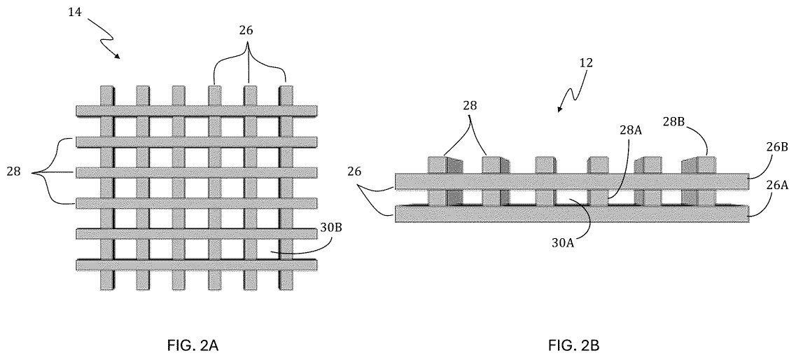

FIG. 1 is a flow chart of method 10 for forming SLIT3 protein-releasing composite scaffolds with hierarchically porous structures. FIG. 1 shows the steps of (1) nanocomposite solution preparation, (2) mold fabrication, (3) nanocomposite casting, (4) mold removal, (5) polydopamine (PDA) deposition, and (6) SLIT3 protein mobilization. FIG. 1 further shows nanocomposite solution 12, mold 14, platform 16, intermediate scaffold 18, polydopamine 20, composite scaffold 22, and SLIT3 protein 24. FIGS. 2A and 2B are top and side views, respectively, of mold 14 used in the fabrication of the composite scaffold 22. FIGS. 1, 2A, and 2B are discussed together herein.

Nanocomposite solution 12 is used to form intermediate scaffold 18 on mold 14 via a casting and solvent evaporation method. Intermediate scaffold 18 comprises a polymer matrix with nanoparticles dispersed therein. Intermediate scaffold 18 is surface modified with polydopamine 20 to form composite scaffold 22. SLIT3 protein 24 can be provided to composite scaffold 22 for bone tissue engineering (BTE) applications. SLIT3 protein 24 can bind to polydopamine 20.

Nanocomposite solution 12 is prepared in step 1. Nanocomposite solution 12 can be water insoluble. Nanocomposite solution 12 can comprise a biodegradable and biocompatible polymer (forming the matrix of intermediate scaffold 18 and composite scaffold 22) and biocompatible nanoparticles dispersed therein. In one non-limiting embodiment, nanocomposite solution 12 can comprise poly(lactic-co-glycolic acid) (PLGA) and hydroxyapatite (HA) nanoparticles (HANPs). PGLA is biocompatible and has controllable biodegradability. HA is biocompatible, hydrophilic, mechanically strong, osteoconductive, and mimics the natural bone's inherent inorganic composition. The incorporation of organic and inorganic components reflects the natural bone extracellular matrix (ECM). The inclusion of organic components (e.g., PGLA) mimics the collagen and proteins found in bone, while the inorganic components (e.g., HANPs) emulate the mineralized aspect of bone tissue. Consequently, the scaffold can successfully support growth and proliferation of mesenchymal stem cells within its microstructure.

The polymer can be dissolved in chloroform with stirring at room temperature. Nanoparticles can be added to the solution and evenly dispersed using probe sonication. Nanoparticles can have a particle size of less than 200 nm. In one non-limiting embodiment, the ratio of polymer to nanoparticles can be 1:1 wt %. In other embodiments, the amount of polymer can exceed the amount of nanoparticles to meet a desired elastic modulus in the resulting composite scaffold 22. For example, the polymer matrix can comprise a ratio of hydroxyapatite nanoparticles to polymer of about 1:1 to about 3:4 wt %.

Scaffolds comprising PGLA and HANPs have shown considerable promise and are the basis for development of composite scaffold 22 disclosed herein. However, it will be recognized by one of ordinary skill it the art, that the method disclosed herein can be used to produce composite scaffolds that include other biocompatible and biodegradable polymer matrices and/or nanoparticles capable of meeting mechanical property requirements and suitable for cell growth.

Mold 14 can be fabricated in step 2. Step 2 can be conducted simultaneously with, prior to, or following nanocomposite solution preparation (step 1). Mold 14 is a 3D sacrificial mold used to form composite scaffold 22. Mold 14 does not form a portion of composite scaffold 22. Mold 14 can be fabricated by 3D printing using conventional 3D printing technology. As shown in FIG. 1, mold 14 can be printed on platform 16. 3D printing technology offers adaptable design options and precise geometrical control, however, other known additive manufacturing processes or casting techniques may be used to fabricate mold 14.

Mold 14 can be formed of a water-soluble material, including but not limited to polyvinyl alcohol (PVA). Use of a water-soluble material can improve ease of fabrication of composite scaffold 22, allowing for removal of mold 14 from intermediate scaffold 18 with water. Other dissolvable materials can be used to form mold 14. Materials soluble in solvents other than water may be suitable provided the solvents and mold material are non-toxic and/or do not leave a residue or otherwise impact the biocompatibility of intermediate scaffold 18 formed thereon.

Mold 14 can have a mesh-like structure as illustrated in FIGS. 2A and 2B. FIGS. 2A and 2B show a rectangular mesh formed by a plurality of perpendicularly intersecting bars 26 and 28, which define rectangular pores 30A and 30B. Bars 26 and 28 can be rectangular prisms provided in a stacked arrangement. As shown in FIG. 2B, mold 14 can include two or more mesh layers, each layer including a plurality of bars 28 disposed on top of and perpendicular to a plurality of bars 26. As shown in FIG. 2B, a first mesh layer can include bars 26A and 28B and a second mesh layer can include bars 26B and 28B. The mesh layers can be arranged in a stacked configuration with alignment of bars 26A and 26B and alignment of bars 28A and 28B, forming connected vertical pores 30A and lateral pores 30B. In one non-limiting embodiment, adjacent bars 26 and adjacent bars 28 can be spaced approximately 500 μm apart, creating pores (i.e., 30B) with square openings of 500 μm×500 μm. Pores 30A and 30B form interconnected and tortuous macropores in composite scaffold 22. The height and spacing of bars 26 and 28 can be selected to provide vertical and lateral pores 30A, 30B of equal or unequal cross-sectional areas. Multiple mesh layers can be combined to increase a thickness or height of mold 14 and thereby composite scaffold 22. In some embodiments, the mesh size (e.g., size of pores) of individual layers can vary.

Composite scaffold 22 can generally take the shape of mold 14 as described further herein. Mold 14 is designed to provide the macrostructure of composite scaffold 22, including interconnected, hierarchical, and tortuous porosity provided, for example, by pores 30A and 30B. Mold 14 is not limited to the embodiment shown. Mold 14 can have other shapes and configurations. Furthermore, the shape and size of pores can vary within mold 14 to provide a desired hierarchical porosity. It will be understood by one of ordinary skill in the art that mold 14 can be designed to provide a structure and hierarchical porosity identified for optimal bone tissue growth.

Intermediate scaffold 18 can be cast on mold 14 via a casting and solvent evaporation method in step 3. Intermediate scaffold 18 comprises a polymer matrix (e.g., PGLA) with nanoparticles (e.g., HANPs) dispersed therein. Intermediate scaffold 18 is also referred to herein as “PGLA-HANPs” scaffold. As illustrated in FIG. 1, nanocomposite solution 12 can be dropwise deposited on mold 14 and allowed to dry. Nanocomposite solution 12 can be dropwise cast on mold 14 in multiple steps. Each step can include covering a surface of mold 14 with nanocomposite solution 12 and allowing the chloroform or solvent to evaporate. Layers of the nanocomposite material comprising the polymer matrix with nanoparticles dispersed therein are added with each step until a desired material thickness is reached. Mold 14 can be gently shaken or vibrated to remove extra nanocomposite solution 12 obstructing pores (e.g., pores 30A and 30B). Nanocomposite solution 12 can be air-dried on mold 14.

Once nanocomposite solution 12 is fully dried, mold 14 can be removed to form intermediate scaffold 18 in step 4. Mold 14 coated with the nanocomposite material can be dissolved in water or other solvent 19 capable of dissolving mold 14 without dissolving or otherwise damaging the resulting intermediate scaffold 18. Mold 14 can be centrifuged or otherwise agitated in water or other solvent to increase the rate of dissolution. The time required for complete dissolution of mold 14 can vary depending on the size of mold 14. Intermediate scaffold 18 can substantially retain its shape and material properties in the mold removal process. The incorporation of nanoparticles (e.g., HANPs) in the polymer matrix enhances mechanical integrity of intermediate scaffold 18. Some nanoparticles may leach from the polymer matrix during mold removal leaving small pores within and open to a surface of in intermediate scaffold 18, which can produce a desired network for 3D cell growth. Additionally, surface roughness on mold 14 can produce small pores open to the surface of intermediate scaffold 18.

Intermediate scaffold 18 formed according to the methods disclosed herein can have a hierarchically porous structure with significant variability in pore size and including optimum pore sizes to promote cell adhesion, proliferation, cell-cell interactions, and osteoblast differentiation. As used herein, the term “hierarchical porosity” refers to interconnected pores of differing size. The hierarchical porous structure is mimetic for the complex extracellular matrix of real bone and can provide improved biomechanical compatibility for bone regeneration. Large pores correspond to inherent pores in mold 14 and void spaces created by mold removal. Small pores are produced by removing residuals of mold 14 and/or leaching of nanoparticles. The average porosity and pore size of intermediate scaffold 18 and final composite scaffold 22, formed by the methods disclosed herein, can closely resemble the porosity of trabecular bone. For example, in a non-limiting embodiment, composite scaffold 22 can have a porosity of around 60-65% and average pore size of 130 to 220 μm.

Once mold 14 has been removed, surfaces of intermediate scaffold 18 can be surface modified with polydopamine 20 in step 5 to form composite scaffold 22 (also referred to herein as “PLGA-HANP-PDA” scaffold). PGLA is hydrophobic, which can limit cell adhesion. Polydopamine 20 can alter surface properties of intermediate scaffold 18, such as roughness and wettability, that can improve material-cell adhesion. Polydopamine 20 is biocompatible and hydrophilic and can facilitate protein loading and release and immobilize SLIT3 protein 24 on composite scaffold 22. A polydopamine surface coating can be formed on intermediate scaffold 18 using dopamine self-polymerization by soaking intermediate scaffold 18 in a solution of dopamine under specific and basic conditions as described further herein. Intermediate scaffold 18 can be shaken in the dopamine solution or the dopamine solution can otherwise be agitated to improve polymerization. Loosely attached or unbounded polydopamine molecules can be removed from the surfaces of composite scaffold 22 following polymerization by washing the composite scaffold 22 with deionized water. Intermediate scaffold 18 can be functionalized with polydopamine 20 according to the disclosed method without altering biocompatibility of composite scaffold 22.

Composite scaffold 22 can be a stable and homogeneous nanocomposite of PLGA-HANPs, functionalized with polydopamine, which can provide superior biomimetic microenvironment of natural bone in terms of matching mechanical properties, biocompatible composition, and bioactive surface. The biomimetic properties of composite scaffold 22 can promote cell adhesion, encourage proliferation and osteogenic differentiation, and facilitate controlled protein loading and release.

The addition of polydopamine 20 can reduce degradation of composite scaffold 22 in a culturing media by blocking hydrolysis of PLGA or other polymer. The biodegradation behavior of composite scaffold 22 is critical in bone tissue regeneration and impacts the associated biological activities. Many factors, such as material composition, porosity, microstructure, and hydrophilicity, can alter the decomposition or degradation rate. It will be understood by one of ordinary skill in the art that the degradation can be optimized by adjusting these parameters within application of the disclosed method. The resulting waste of decomposition of the disclosed material is biocompatible. The biocompatibility of waste is necessary to maintain an integral support for cell growth and avoid any resultant toxicity. Surface modification with polydopamine 20 can additionally promote salt deposition from the culturing media, which can further depress degradation. The slower degradation of composite scaffold 22 can provide growing cells more time to build denser bone tissue at later stages of mesenchymal stem cell culture differentiation.

In step 6, composite scaffold 22 is incorporated with SLIT3 protein 24. SLIT3 protein 24 can promote bone formation and control the resorption process in natural bone healing. Composite scaffold 22 can be prepared for surface SLIT3 protein 24 incorporation. Composite scaffold 22 can be incubated in a solution of SLIT3 protein 24 over a period of time to initiate and maintain protein bonding with polydopamine 20 on the surface of composite scaffold 22. SLIT3 protein, along with the physical and chemical properties of composite scaffold 22, establishes adhesion, support proliferation, and osteogenic differentiation of bone marrow derived mesenchymal stem cells.

### Example

### Scaffold Fabrication

A 3D nanocomposite scaffold with a hierarchically porous structure was fabricated on a 3D-printed water-dissolvable PVA mold using a pre-prepared composite solution and the casting and solvent evaporation method. A mesh-like mold was designed using the COMSOL software package. The mold was designed as replicated blocks, with each block composed of two layers of straight lines using rectangular prisms with a length of 10 mm and each layer's lines perpendicular to the next layer's lines. The lines in the first layer were parallel to the x-axis, whereas those of the second layer were aligned with the Y-axis. The distance between each adjacent line was 500 μm. This configuration created vertical and lateral square-opening inherent pores of 500 μm×500 μm in the mold structure. The design was printed using PVA water-soluble filaments and an Ender 5 Printer from CREALITY. The printing parameters of 50 mm/s printing speed, 220° C. nozzle temperature, 60° C. bed temperature, and 0.18 mm for the layer height were followed to print the mesh-like PVA mold with dimensions of 10 mm width, 9 mm length, and 2.7 mm height.

A water non-dissolvable nanocomposite was produced by dissolving 100 mg of PLGA in 4 mL chloroform under stirring at room temperature for three hours until complete dissolution. 100 mg of HANP powder was added to the solution and evenly dispersed using probe sonication with an amplitude of 50% for 150 seconds. The printed mold was subjected to dropwise casting using 200 μL, 300 μL, and 400 μL of the prepared solution on the PVA mold. The casting was conducted within five steps, each involving total coverage for the mold, then waiting for five minutes for chloroform evaporation. The extra material obstructing the inherent pores was removed by gently shaking the coated mold, and the resultant structure was allowed to air-dry for four hours at room temperature, producing a white solid PLGA-HANPs composite structure on the PVA mold. The coated mold was centrifuged at 400 rpm in tap water for two hours to remove the PVA sacrificial mold entirely, producing the PLGA-HA NPs hierarchically porous scaffold.

A biocompatible and hydrophilic polydopamine layer was used to facilitate protein loading and release and immobilize the SLIT3 protein on the scaffold. The polydopamine surface coating was created using dopamine self-polymerization by soaking the scaffold in a solution of 10 mg/mL of dopamine in 10 mM of Tris-HCl with a pH of 8.0 under shaking at 100 rpm for six hours at 37° C. The surface-modified scaffold was washed three times with deionized water after polymerization to remove the loosely attached PDA molecules from the scaffold surface. The resulting gray and brown PDA-coated scaffold was prepared for surface SLIT3 protein incorporation (FIG. 1-g). The scaffold was incubated in a solution of SLIT3 (1 μg/mL in 1×PBS) overnight at 4° C. to initiate and maintain protein bonding with polydopamine on the scaffold surface.

### Primary Cell Culturing, Harvesting, and Scaffold Seeding

Primary stem cell culturing was performed according to the instructions provided by the supplier. Stem cells were grown in T-75 flasks pre-coated with bovine plasma fibronectin and kept at 37° C. and a 5% CO₂ environment for 24 hours. The fibronectin solution was completely replaced with 20 mL of media the next day before adding cells. The cells were grown until they reached confluence, after which they were detached using trypsin, harvested, counted, and prepared for seeding into the scaffold's matrix with three cell suspension densities. Four scaffolds were sterilized before cell seeding by soaking them in 70% ethanol for two hours in a biosafety cabinet. Three experimental scaffolds were loaded with the SLIT3 protein by adding 100 μL of protein solution (1 μg/mL concentration) to the scaffold surface in a 24-well culture plate, while the fourth scaffold served as a control. Three cell densities of 2000, 4000, and 5000 cells/cm2 in a total volume of 1 mL were tested. These cellular suspensions were seeded to the surface of the experimental and control scaffolds and incubated at 37° C. and a 5% CO₂ environment for 72 hours. The culture medium was replenished every three days, and cell proliferation was monitored for 6-12 days using the FLoid™ Cell Imaging Station (ThermoFisher scientific).

### Characterization of the PLGA-HANPs-PDA Scaffold

The microstructure, pore size and hierarchical porosity, and morphology of the optimized PLGA-HANPs-PDA scaffold's cellular adhesion were obtained with scanning electron microscopy (SEM) using an FEI Quanta instrument, US. SEM analysis was also used to study and confirm the microstructural modification of the scaffold's surface and the PLGA-HANPs-PDA cross-section. The cell-seeded scaffold was directly collected from the culture plate, immersed in 2.5% glutaraldehyde solution for cell fixation, then washed with DI water three times to remove unattached cells and culture media supernatant. The scaffold was then placed back into the culture plate to allow it to dry at room temperature. All scaffolds, with and without cells, were coated with a ˜5 nm carbon thin layer and mounted on aluminum stubs before SEM analysis. Pore size and HANPs diameter were measured using ImageJ software after applying specific linear adjustments to the acquired SEM images at 1 mm scale and 20 μm, respectively.

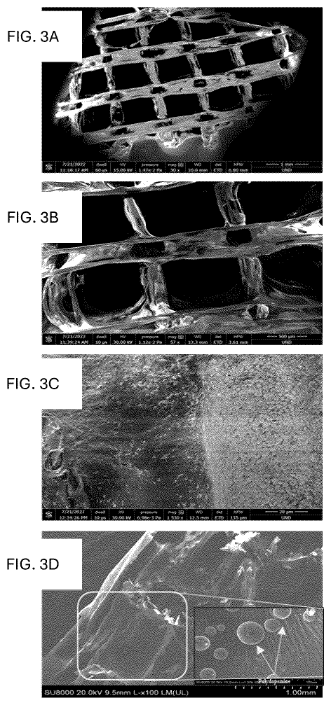

The SEM micrographs of the surface and cross-sectional microstructure of the PLGA-HANPs-PDA scaffold at different magnifications are depicted in FIGS. 3A-3C. PLGA-HANPs-PDA scaffold exhibited a hierarchically porous structure with significant variability in pore size and the successful polymerization of PDA on the surface. The high pore size corresponded to the inherent pores in the mesh-like mold and the void spaces created by mold strand removal. The small pores were produced by removing the residuals of the PVA mold from the coating composite material. Incorporating HANPs into the PLGA matrix enhanced the mechanical integrity of the composite. FIG. 3C illustrates the confirmation of successful polymerization of dopamine on the PLGA-HANPs surface.

Polydopamine functionalization was also confirmed by addressing the rougher surface of the scaffold FIGS. 3A-3D; however, the self-polymerization of dopamine on the scaffold surface introduced minor cracks (FIGS. 3A and 3B). Inherent and multi-leveled intersectional pores were observed with an average size of 178.07±45 μm (FIG. 3A). The cross-sectional view of the scaffold is illustrated in FIG. 3D. The PDA functionalization was also confirmed through the cross-sectional view of the PLGA-HANPs-PDA; however, the deposition density there was far higher on the surface (3C) than the cross-section (FIG. 3D) since the scaffold's surface is more exposed to the dopamine monomers that involved in the self-polymerization.

The mechanical properties of the PLGA-HANPs-PDA scaffold was evaluated with a compression test using 200 μL, 300 μL, and 400 μL of the corresponding casting solution. This step aimed to evaluate the casting process and how the scaffold's mechanical strength would be related to the casting solution's composition and volume. The scaffold with a surface area of 90 mm² and thickness of 2.7 mm was tested in dry conditions at room temperature. The computerized universal testing machine (Shimadzu AGS-X) equipped with a 5 kN loading cell was used to obtain the stress/strain curves. The testing procedure was standardized to ensure consistency. Stress/strain curves were acquired while applying compression at a speed of 1 mm/min, with a maximum compression force of 150 N. The compression modulus at each composition and volume was calculated from the linear region of the obtained curves. The elastic modulus will have a decisive contribution to the expected biological activities of the cells. The measurements and elastic moduli were repeated in triplicate and presented in the form of average±standard deviation.

The hierarchically porous architecture of the PLGA-HANPs-PDA scaffold is more mimetic for the complex extracellular matrix of the real bone. The PLGA-HANPs-PDA scaffolds formed with 300 μL and 400 μL casing solution each demonstrated a compression strength with a modulus greater than 0.4 MPa, which is suitable to withstand and support cell culturing and other in vitro biological activities. The PLGA-HANPs-PDA scaffold formed with 400 μL of casting solution had a compression strength with a modulus of 0.58±0.05 MPa, which was greater than the 300 μL scaffold. HANPs addition and PDA coating both were shown in enhance the scaffold's compression mechanical strength compared to PGLA. This trend highlights the accessibility to improve the elastic modulus by the addition of HANPs, PDA surface functionalization, and controlling the casting volume of the polymeric nanocomposite.

The optimized scaffold's porosity ratio was assessed by determining the density of the PLGA-HANPs-PDA scaffold and the apparent density of bulk PLGA-HANPs-PDA. The masses and volumes of the bulk and scaffold forms were used to calculate the apparent density of the composite material and the scaffold. The volume of the bulk was directly calculated from the measured dimensions, where the volume of the scaffold would equal the mold volume determined from the design software. The porosity ratio was defined after acquiring the densities according to the following formula:

$$Porosity ( \% ) = ( 1 - \frac{{ρ}_{scaffold}}{{ρ}_{b u l k}} ) * 100$$

where ρ_{scaffold} is the apparent density of the optimized PLGA-HANPs-PDA scaffold and ρ_{bulk} is the density of the bulk casting material. The PLGA-HANPs-PDA scaffold exhibited an average porosity of 63.73±3.94% with an average pore size of 178.07±45.01 μm, as reported in the microstructural properties.

### SLIT3 Protein Release Profile

SLIT3-loaded scaffolds were washed with pure PBS three times to remove the loosely attached protein from the functionalized surface. The scaffold was immersed in 1.2 mL pure PBS to stimulate the protein release from the surface. The release profile was tracked at six time points such that three samples of 100 μL PBS containing SLIT3 protein were collected after 1, 2, 5, 7, 9, and 10 hour(s) of the initial immersion. Each batch of samples was collected from the scaffold PBS solution and directly replaced with an equivalent volume of PBS. The scaffold protein release profile was acquired using a His-tag Protein ELISA kit (Abcam, USA), which is designed to quantify proteins with a C-terminal 6-his-tag peptide sequence, as on the surface of the SLIT3 protein. The manufacturer's instructions were followed precisely throughout the standard dilution, reagent mixing, and incubation. The percentage release calculation was determined using the following formula:

$$\% SLIT 3 release = \frac{cumulative amount of SLIT 3 released in collected sample}{total amount of SLIT 3 immobilized on the PLGA - H A N P s - PDA scaffold} * 100$$

The SLIT3 protein release profile in the PBS solution was investigated and tracked. The sample collected after one hour exhibited a high and rapid protein release, approximately 22%, due to the dense deposition of the SLIT3 protein on the scaffold surface. The next samples taken after two and five hours experienced relatively slow and varying release. Specifically, the released protein reached 35% after two hours and took more than three hours to increase by 10% in the third sample. The release for samples four and five needed two hours to increase from 45% to 51% and two more hours to achieve 58%, respectively. The final accumulated amount of the released protein in the last sample reached 62% of the original deposited protein after ten hours of scaffold immersion in PBS. It was necessary to assess the associated release profile from the scaffold surface to the phosphate buffer solution (PBS) before proceeding to the cellular testing. The delicate execution of this step was of utmost importance since our hypothesis regarding the SLIT3 protein's efficacy relies on the precise attachment of the protein to the scaffold surface and this would in turn determine how the release will influence cellular attachment, proliferation and differentiation. The gradual release of the SLIT3 protein from the scaffold surface is essential for more precise and direct manipulation of the biological activities at the cellular level. The developed scaffold exhibited a gradual protein release profile that will support the thriving of MSCs by exploiting the bioactivity of SLIT3 in more efficient manner.

### Cell Culturing, Harvesting, and Seeding

The cultured cells were harvested after they densely spread over the T-75 fibronectin-coated flask after three days of incubation, with 93.5% viability. The cell line was ready for further passages as needed at the end of the primary culturing.

The secondary cell culture was applied by seeding the cells in the PLGA-HANPs-PDA scaffold after the cell line was established. The scaffold must exhibit high biocompatibility and provide the essential conditions for cells to attach and thrive on its surface during cell culturing to achieve optimal results. The MSCs seeded in the experimental scaffold were assessed at various time intervals spanning a duration of one week. The cells that were initially suspended in the culture media of the experimental scaffolds appeared in rounded morphology at day 0. Observations on day 3 revealed that the cells clearly grew and successfully adhered to the scaffold surface and the culturing flask at the three culturing densities. Suspended sphere-shaped cells were transformed to a spindle-like shape in a high density pattern, indicating promoted ECM-Cell interactions. The cells maintained consistent behavior during the next four days as the cells became denser and more accumulated due to enhanced cellular signaling and communication. This finding emphasizes the scaffold's ability to experimentally support various cell culturing densities without compromising biocompatibility while highlighting the role of the SLIT3 protein and gradual protein release profile in supporting stem cell proliferation within the scaffold for the seven days of culturing.

The control scaffold, without the SLIT3 protein, was also seeded with a cell suspension of 5000 cells/cm2 density and then incubated under standard conditions at a temperature of 37° C. and a CO₂ level of 5% for a duration of 12 days. Significantly, the cells demonstrated limited population growth in the absence of the SLIT3 protein, in contrast to the situation when the SLIT3 protein was present in the experimental scaffolds. The cell population dropped sharply after 3, 6 and 12 days, respectively, as the cells did not adhere to the scaffold. This outcome supports the SLIT3 protein's efficacy in promoting mesenchymal stem cell adhesion and proliferation within the PLGA-HANPs-PDA scaffold. The SLIT3 protein provided the crucial structural support for cell adhesion, proliferation, migration, and homing in the experimental scaffold.

### Cell Adhesion

MSC adhesion on the scaffold surface was visualized using Scanning Electron Microscopy (SEM) after seven days of cell culture. The SLIT3-free samples were discouraging for further SEM analysis due to extremely low cell growth as confirmed in the bright field microscopy. The seeded cells were observed to be attached and randomly distributed on the PLGA-HANPs-PDA scaffold surface, confirming SLIT3 absorption on the scaffold surface, effective protein bioactivity, and scaffold biocompatibility. The stem cells' successful and promoted attachment can be inferred by the grown microvilli and pseudopodia observed on the cell surfaces, which extended through the scaffold at different focal adhesion points; however, cell aggregation was also noticed due to the interferential meshing of these actin filaments of the adjacent dead cells. The PLGA-HANPs-PDA scaffold had initiated degradation spots throughout the scaffold structure after seven days of culturing. These spots originated from cell proliferation and the related biological activities between the scaffold, cells, and culture media. Cell adhesion on the surface validates the synergistic effect of the compositional, structural, and functional properties of the PLGA-HANPs-PDA scaffold, allowing it to mimic native ECM.

The indirect 3D printing technique was used to produce a hierarchically porous and bone ECM mimicking SLIT3-releasing composite scaffold. As disclosed herein, the composite scaffold synthesized from the nanocomposite of PLGA-HANPs-PDA supports protein adsorption, release, and subsequent MSCs cell culture. Characterization results confirmed consistently homogenous and thermally stable nanocomposite of PLGA-HANPs functionalized with PDA without biocompatibility alteration. The matching structural and mechanical properties sustained fulfilling biodegrading and gradual protein release that will affect the subsequent cellular testing. Bone marrow derived MSCs were growing within the scaffold microstructure at three different culturing densities. The disclosed SLIT3-releasing composite scaffold exhibits remarkable bone biomimetic properties due to its tailored composition and intricate structure and significantly promotes cell adhesion, proliferation, and differentiation.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

Any relative terms or terms of degree used herein, such as “substantially”, “essentially”, “generally”, “approximately” and the like, should be interpreted in accordance with and subject to any applicable definitions or limits expressly stated herein. In all instances, any relative terms or terms of degree used herein should be interpreted to broadly encompass any relevant disclosed embodiments as well as such ranges or variations as would be understood by a person of ordinary skill in the art in view of the entirety of the present disclosure, such as to encompass ordinary manufacturing tolerance variations, incidental alignment variations, transient alignment or shape variations induced by thermal, rotational or vibrational operational conditions, and the like. Moreover, any relative terms or terms of degree used herein should be interpreted to encompass a range that expressly includes the designated quality, characteristic, parameter or value, without variation, as if no qualifying relative term or term of degree were utilized in the given disclosure or recitation.

1. A polymeric scaffold for bone repair and regeneration, the polymeric scaffold comprising:
a body comprising:
a biodegradable polymer matrix; and
nanoparticles dispersed in the biodegradable polymer matrix; and
a polydopamine surface coating on the body.

2. The polymeric scaffold of claim 1, wherein each of the biodegradable polymer matrix and the nanoparticles are biocompatible.

3. The polymeric scaffold of claim 1, wherein the biodegradable polymer is poly(lactic-co-glycolic acid).

4. The polymeric scaffold of claim 3, wherein the nanoparticles are hydroxyapatite.

5. The polymeric scaffold of claim 4, wherein the nanoparticles have a particle size less than 200 nm.

6. The polymeric scaffold of claim 1, wherein the polymer matrix comprises a ratio of hydroxyapatite nanoparticles to polymer of 1:1 to 3:4 wt %.

7. The polymeric scaffold of claim 1 and further comprising Slit Guidance Ligand 3 (SLIT3) protein disposed on the polydopamine surface coating.

8. The polymeric scaffold of claim 1, wherein the body has a mesh-like structure.

9. The polymeric scaffold of claim 1, wherein the body comprises multiple mesh-like layers disposed in a stacked arrangement.

10. The polymeric scaffold of claim 1, wherein the body comprises hierarchical and interconnected porosity.

11. The polymeric scaffold of claim 10, wherein an average pore size is between about 133 μm and 223 μm.

12. The polymeric scaffold of claim 1 having a compression modulus of greater than 0.4 MPa.

13. A method of forming a composite scaffold for bone tissue engineering, the method comprising:
preparing a solution comprising a biodegradable polymer and a plurality of nanoparticles in a solvent;
applying the solution to a surface of a mold and drying the solution to form a polymer matrix having nanoparticles dispersed therein;
removing the mold from the polymer matrix to form an intermediate scaffold; and
applying a polydopamine coating to the intermediate scaffold to form the composite scaffold.

14. The method of claim 13, and further comprising providing SLIT3 protein on the polydopamine coating.

15. The method of claim 14, wherein the biodegradable polymer is poly(lactic-co-glycolic acid).

16. The method of claim 14, wherein the nanoparticles are hydroxyapatite.

17. The method of claim 16, wherein the polymer matrix comprises a ratio of hydroxyapatite nanoparticles to polymer of 1:1 to 3:4 wt %.

18. The method of claim 13, wherein the mold is 3D printed and has a layered mesh-like architecture.

19. The method of claim 13, wherein removing the mold comprises dissolving the mold.
